(19) United States
(12) Patent Application Publication
(10) Pub. No.: US 2025/0289846 A1
(43) Pub. Date: Sep. 18, 2025

(54) METHODS OF PURIFYING ANTIBODY-NUCLEIC ACID CONJUGATES

(71) Applicant: Bruker Spatial Biology, Inc., Seattle, WA (US)

(72) Inventors: Brian J. FILANOSKI, Vashon, WA (US); Gary K. GEISS, Seattle, WA (US)

(21) Appl. No.: 18/860,126

(22) PCT Filed: Apr. 27, 2023
(86) PCT No.: PCT/US2023/020105

§ 371 Date: Oct. 25, 2024

Related U.S. Application Data
(60) Provisional application No. 63/335,777, filed on Apr. 28, 2022.

(51) Int. Cl. C07K 1/14; C12N 15/10

(52) U.S. Cl. CPC C07K 1/145; C12N 15/1003

(57) ABSTRACT

The present disclosure provides methods and kits directed to the purification of antibody-nucleic acid conjugates using a PEG-based precipitation step.

### RELATED APPLICATIONS

This application claims priority to, and the benefit of, U.S. Provisional Application No. 63/335,777, filed on Apr. 28, 2022, the contents of which are incorporated herein by reference in their entireties.

### BACKGROUND

Antibody-nucleic acid conjugates are useful in a variety of contexts, including, but not limited to, therapeutic applications in which antibodies are used to specifically target anti-sense oligonucleotides (ASOs) to particular cells (e.g. see Dugal-Tessier et al. J Clin Med. 2021 Feb. 18;10(4):838) as well as diagnostic and research applications in which the antibody-nucleic acid conjugates are used to detect target analytes within biological samples (e.g. see He et al. bioRxiv 2021 Nov. 3, 467020, doi: 10.1101/2021.11.03.467020). Importantly, all of these uses require highly pure antibody-nucleic acid conjugates that are substantially free of contaminants, most notably excess free nucleic acid. Accordingly, there is a need in the art for improved, efficient, and cost-effective methods and kits directed to the purification of antibody-nucleic acid conjugates. The present disclosure addresses this need.

### SUMMARY

The present disclosure provides methods for purifying antibody-nucleic acid conjugates from a solution including the antibody-nucleic acid conjugates and unconjugated nucleic acid, the methods comprising: a) contacting the solution and a purification solution including polyethylene glycol (PEG) and sodium chloride (NaCl) to produce a mixture including antibody-nucleic acid conjugates, unconjugated nucleic acids, PEG and NaCl such that the concentration of PEG in the mixture is between about 5% to about 30% (w/v) and the concentration of NaCl in the mixture is between about 100 mM to about 250 mM; b) centrifuging the mixture to produce a supernatant and a pellet; c) isolating the pellet from the supernatant; d) resuspending the pellet with a resuspension solution to produce a purified solution; e) repeating steps (a)-(d) at least once using the purified solution of step (d) to produce a purified solution that is at least 90% pure for the antibody-nucleic acid conjugates, as measured by size exclusion (SEC) high performance liquid chromatography (HPLC). In some aspects, the concentration of PEG in the mixture is between about 10% to about 25% (w/v). In some aspects, the concentration of NaCl in the mixture is between about 150 mM to about 200 mM.

In some aspects of the preceding methods, purified solution is at least 95% pure for the antibody-nucleic acid conjugates, as measured by size exclusion (SEC) high performance liquid chromatography (HPLC).

In some aspects of the preceding methods, step (b) comprises centrifuging the mixture at a speed of at least about 1,000 RCF (xg).

In some aspects of the preceding methods, the centrifuging in step (b) is performed for at least about 2 minutes.

In some aspects of the preceding methods step (a) further comprises incubating the mixture for about 20 to about 40 minutes at about 20° C.

In some aspects of the preceding methods, step (d) further comprises incubating the resuspended pellet for about 20 to about 40 minutes at about 37° C.

In some aspects of the preceding methods, step (a) further comprises vortexing the mixture.

In some aspects of the preceding methods, the resuspension solution comprises phosphate buffered saline (PBS).

In some aspects of the preceding methods, the PEG is: i) PEG-6000; ii) PEG-8000; iii) PEG-10000; or iv) PEG-35,000. In some aspects, the PEG is PEG-6000. In some aspects, the PEG is PEG-8000.

In some aspects of the preceding methods, contacting the solution and a purification solution in step (a) comprises contacting the solution and the purification solution at a ratio of 1:1 (v/v).

In some aspects of the preceding methods, the concentration of PEG in the purification solution is between about 35% to about 45% (w/v). In some aspects of the preceding methods, the concentration of PEG in the purification solution is about 40% (w/v).

In some aspects of the preceding methods, the concentration of NaCl in the purification solution is between about 150 mM to 250 mM. In some aspects of the preceding methods, the concentration of NaCl in the purification solution is about 200 mM.

In some aspects of the preceding methods, the antibodies comprise a Fab, a Fab′, a F(ab′)2, a Fv fragment, a single-chain (sc)Fv (scFv) antibody fragment, a linear antibody, a single domain antibody (sdAb), a camelid VHH domain, a multi-specific antibody or any combination thereof.

In some aspects of the preceding methods, the nucleic acids are single-stranded, double-stranded or partially double-stranded.

In some aspects of the preceding methods, the nucleic acids comprise L-DNA. In some aspects of the preceding methods, the nucleic acids comprise D-DNA. In some aspects of the preceding methods, the nucleic acids comprise a combination of L-DNA and D-DNA.

In some aspects of the preceding methods, the method is performed in one or more wells of a multi-well plate, thereby simultaneously purifying a plurality of antibody-nucleic acid conjugates.

In some aspects of the preceding methods, the method is performed in two or more wells of a multi-well plate, thereby simultaneously purifying a plurality of antibody-nucleic acid conjugates in distinct wells.

The present disclosure provides methods of producing antibody-nucleic acid conjugates, the methods comprising: a) contacting a first solution comprising nucleic acids including at least one reactive group and a second solution including antibodies under conditions such that the at least one reactive group of the nucleic acids reacts with the antibodies, thereby forming antibody-nucleic acid conjugates; b) purifying the antibody nucleic acid conjugates using any one of the purification methods described herein. In some aspects, the at least one reactive group comprises a maleimide moiety.

The present disclosure provides kits comprising: i) a first solution including nucleic acids including at least one reactive group; and ii) a purification solution including polyethylene glycol (PEG) and sodium chloride (NaCl).

In some aspects of the preceding kits, the nucleic acids are single-stranded, double-stranded or partially double-stranded.

In some aspects of the preceding kits, the nucleic acids comprise L-DNA. In some aspects of the preceding kits, the nucleic acids comprise D-DNA. In some aspects of the preceding kits, the nucleic acids comprise a combination of L-DNA and D-DNA.

In some aspects of the preceding kits, the at least one reactive group comprises a maleimide moiety.

In some aspects, the preceding kits further comprise a second solution including antibodies.

In some aspects of the preceding kits, the antibodies comprise a Fab, a Fab′, a F(ab′)2, a Fv fragment, a single-chain (sc)Fv (scFv) antibody fragment, a linear antibody, a single domain antibody (sdAb), a camelid VHH domain, a multi-specific antibody or any combination thereof.

In some aspects the preceding kits further comprise a third solution including nucleic acids including at least one reactive group, wherein the nucleic acids in the first solution include a nucleic acid sequence that is different from the nucleic acid sequence of the nucleic acids in the third solution.

In some aspects of the preceding kits, the PEG is: i) PEG-6000; ii) PEG-8000; iii) PEG-10000; or iv) PEG-35,000. In some aspects, the PEG is PEG-6000. In some aspects, the PEG is PEG-8000.

In some aspects of the preceding kits, the concentration of PEG in the purification solution is between about 35% to about 50% (w/). In some aspects of the preceding kits, the concentration of PEG in the purification solution is about 40% (w/v).

In some aspects of the preceding kits, the concentration of NaCl in the purification solution is between about 150 mM to 250 mM. In some aspects of the preceding kits, the concentration of NaCl in the purification solution is about 200 mM.

In some aspects, the preceding kits further comprise at least one aliquot of TCEP.

In some aspects, the preceding kits further comprise at least one spin filter.

In some aspects, the preceding kits further comprise at least one multi-well plate.

Any of the aspects and/or embodiments described above and herein can be combined with any other aspect and/or embodiment described above and herein.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Throughout the specification, the recitation of ranges of values described as “between x and y” or “between x to y”, wherein x and y are two values, is understood to be inclusive of x and y. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety for all purposes. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

The above and further features will be more clearly appreciated from the following detailed description when taken in conjunction with the accompanying drawings.

FIG. 1 shows a SEC HPLC chromatogram of an unconjugated antibody.

FIG. 2 shows a SEC HPLC chromatogram of an unpurified antibody/nucleic acid conjugation reaction.

FIG. 3 shows a SEC HPLC chromatogram of an antibody/nucleic acid conjugation reaction purified using the methods of the present disclosure wherein no NaCl was used during the purification, resulting in little to no purification of the antibody-nucleic acid conjugate.

FIG. 4 shows a SEC HPLC chromatogram of an antibody/nucleic acid conjugation reaction purified using the methods of the present disclosure wherein NaCl was used during the purification, resulting in purification of the antibody-nucleic acid conjugate to a purity level over 90%.

FIG. 5 shows a SEC HPLC chromatogram of two antibody/nucleic acid conjugation reactions purified using the methods of the present disclosure and an unpurified antibody-nucleic acid conjugation reaction.

FIG. 6 is a graph comparing the activity of antibody-nucleic acid conjugates purified using either the methods of the present disclosure (y-axis) or existing SEC HPLC methods (x-axis).

FIG. 7A is a graph comparing the activity in NK92 cells of antibody-nucleic acid conjugates purified using a separate format (y-axis) and a pooled format (x-axis) of the methods of the present disclosure.

FIG. 7B is a graph comparing the activity in HCC78 cells of antibody-nucleic acid conjugates purified using a separate format (y-axis) and a pooled format (x-axis) of the methods of the present disclosure.

### DETAILED DESCRIPTION

The present disclosure is based at least in part on methods and kits for purifying antibody-nucleic acid conjugates.

As would be appreciated by the skilled artisan, existing methods of producing and subsequently purifying antibody-nucleic acid conjugates suffer from various disadvantages that limit their usefulness. For example, many existing methods of purifying antibody-nucleic acid conjugates rely on the use of size exclusion chromatography HPLC, which separates antibody-nucleic acid conjugates from free nucleic acids and/or free antibodies based on size. However, SEC HPLC requires expensive equipment, requires specialized training and is not easily scalable (each sample must be processed through the SEC HPLC column individually). Other existing methods of purifying antibody-nucleic acid conjugates use affinity chromatography. However, the affinity matrices used in these methods can be expensive and often require the use of harsh elution conditions that can damage the antibody-nucleic acid conjugates.

In contrast to these existing methods, the present disclosure provides antibody-nucleic acid conjugate purification methods, and associated kits and compositions, that are based on polyethylene glycol (PEG) precipitation. The antibody-nucleic acid conjugate purification methods presented herein are less expensive, faster, and easier to perform as compared to the existing purification methods described, while still providing high levels of purity. Moreover, the methods described herein do not rely on the use of harsh conditions, thereby preventing damage to the antibody-nucleic acid conjugates. Finally, unlike existing methods, the purification methods described herein are scalable and can even be performed in microplates (e.g. 6, 12, 24, 48, 96, 384, 1536, 3456 and 9600 well plates). By being scalable, the purification methods described herein are useful in the labeling of various species of antibodies simultaneously. Such an application is particularly useful within the diagnostic and research context, in which a user may desire to interrogate dozens to hundreds of different molecular targets using dozens to hundreds of distinct antibody-nucleic acid conjugates.

### Purification Methods of the Present Disclosure

The present disclosure provides methods for purifying antibody-nucleic acid conjugates from a solution comprising the antibody-nucleic acid conjugates and unconjugated nucleic acids, the method comprising a) contacting the solution and a purification solution comprising polyethylene glycol (PEG) and sodium chloride (NaCl) to produce a mixture comprising antibody-nucleic acid conjugates, unconjugated nucleic acids, PEG and NaCl; b) centrifuging the mixture to produce a supernatant and a pellet; c) isolating the pellet from the supernatant; d) resuspending the pellet with a resuspension solution to produce a purified solution; e) repeating steps (a)-(d) at least once using the purified solution of step (d) to produce a purified solution.

The present disclosure provides methods for purifying antibody-nucleic acid conjugates from a solution comprising the antibody-nucleic acid conjugates and unconjugated nucleic acids, the method comprising a) contacting the solution and a purification solution comprising polyethylene glycol (PEG) and sodium chloride (NaCl) to produce a mixture comprising antibody-nucleic acid conjugates, unconjugated nucleic acids, PEG and NaCl; b) centrifuging the mixture to produce a supernatant and a pellet; c) isolating the pellet from the supernatant; d) resuspending the pellet with a resuspension solution to produce a purified solution.

As described supra, the purification methods of the present disclosure, unlike existing methods known in the art, is easily scalable, allowing for a plurality of different antibodies species to be labeled simultaneously or nearly simultaneously. To that end, any of the purification methods described herein can be performed in at least one well of a microwell plate, preferably in a plurality of wells in a microwell plate either simultaneously or nearly simultaneously. The purification methods described herein can be performed in multi-tube strips, multi-tube arrays, or similar structures. For ease of description the terms “microwell plate” or “multi-well plate” or “multi-tube array” are used substantially interchangeably herein. The precise structure utilized would depend on the embodiment.

For example, a microwell plate can be a 6-well plate, a 12-well plate, a 48-well plate, a 96-well plate, a 384-well plate, a 1536-well plate, a 3456-well plate or a 9600-well plate.

Accordingly, the present disclosure provides a method of purifying a plurality of antibody-nucleic acid conjugates, the method comprising: a) contacting a solution including antibody-nucleic acid conjugates and unconjugated nucleic acids with a purification solution comprising polyethylene glycol (PEG) and sodium chloride (NaCl) to produce a mixture including antibody-nucleic acid conjugates, unconjugated nucleic acid, PEG and NaCl; b) centrifuging the mixture to produce a supernatant and a pellet; c) isolating the pellet from the supernatant; d) resuspending the pellet with a resuspension solution to produce a purified solution; e) repeating steps (a)-(d) at least once using the purified solution of step (d) to produce a purified solution, wherein steps (a)-(e) are performed in a plurality of wells, thereby producing a plurality of purified solutions. In some aspects of the preceding method, any combination of steps (a)-(e) may be performed simultaneously or nearly simultaneously in a plurality of wells in a multi-well plate. In some aspects of the preceding method, some of the steps (a)-(e) may be performed sequentially in a plurality of wells in a multi-well plate while the remaining steps are performed simultaneously or nearly simultaneously.

Accordingly, the present disclosure provides a method of purifying a plurality of antibody-nucleic acid conjugates, the method comprising: a) contacting a solution including antibody-nucleic acid conjugates and unconjugated nucleic acids with a purification solution comprising polyethylene glycol (PEG) and sodium chloride (NaCl) to produce a mixture including antibody-nucleic acid conjugates, unconjugated nucleic acid, PEG and NaCl; b) centrifuging the mixture to produce a supernatant and a pellet; c) isolating the pellet from the supernatant; d) resuspending the pellet with a resuspension solution to produce a purified solution; wherein steps (a)-(d) are performed in a plurality of wells, thereby producing a plurality of purified solutions. In some aspects of the preceding method, any combination of steps (a)-(d) may be performed simultaneously or nearly simultaneously in a plurality of wells in a multi-well plate. In some aspects of the preceding method, some of the steps (a)-(d) may be performed sequentially in a plurality of wells in a multi-well plate while the remaining steps are performed simultaneously or nearly simultaneously.

In some aspects of the preceding methods, the antibodies of the antibody-nucleic acid conjugates located in different wells can be different. In a non-limiting example, a first well of a multi-well plate may contain antibody-nucleic acid conjugates comprising a first antibody that binds to a first protein target, and a second well of the multi-well plate may contain antibody-nucleic acid conjugates comprising a second antibody that binds to a second protein target, wherein the first protein target and the second protein target are distinct. In some aspects, multiple wells in the multi-well plate may contain solutions comprising antibody-nucleic acid conjugates with the same antibody while other wells in the multi-well plate may contain solutions comprising antibody-nucleic acid conjugates comprising a different antibody. In a non-limiting example, a first well of a multi-well plate may contain antibody-nucleic acid conjugates comprising a first antibody that binds to a first protein target, a second well of a multi-well plate may contain antibody-nucleic acid conjugates comprising the first antibody that binds to the first protein target, and a third well of the multi-well plate may contain antibody-nucleic acid conjugates comprising a second antibody that binds to a second protein target, wherein the first protein target and the second protein target are distinct. In another non-limiting example, a first well of a multi-well plate may contain antibody-nucleic acid conjugates comprising a first antibody that binds to a first protein target, a second well of a multi-well plate may contain antibody-nucleic acid conjugates comprising a second antibody that binds to a second protein target, and a third well of the multi-well plate may contain antibody-nucleic acid conjugates comprising a third antibody that binds to a third protein target, wherein the first protein target, the second protein target and the third protein target are distinct. The skilled artisan would appreciate that in this way, the purification methods described herein allow for the rapid and simultaneous purification of a plurality of antibody-nucleic acid conjugates, wherein the plurality of conjugates comprise a plurality of different conjugate species that bind to a plurality of different molecular targets. Such an application is particularly useful in the diagnostic and research uses described above, wherein the user is often interrogating a biological sample for the presence, absence and/or location of a plurality of different molecular targets.

In some aspects, prior to performing any of the purification methods of the present disclosure, a plurality of antibody-nucleic acid labeling reactions can be pooled together such that a plurality of different antibody-nucleic acid conjugates is concurrently purified in the same solution using the methods of the present disclosure.

In a similar manner, in some aspects of the preceding methods, the nucleic acids of the antibody-nucleic acid conjugates located in different wells of a multi-well plate can be different and/or distinguishable in at least one portion of their sequence. In a non-limiting example, a first well of a multi-well plate may contain antibody-nucleic acid conjugates comprising a nucleic acid including a first nucleic acid sequence, and a second well of the multi-well plate may contain antibody-nucleic acid conjugates including a nucleic acid including a second nucleic acid sequence, wherein the first nucleic acid sequence and the second nucleic acid sequence are distinct. In some aspects, multiple wells in the multi-well plate may contain solutions comprising antibody-nucleic acid conjugates with a nucleic acid including a first nucleic acid sequence while other wells in the multi-well plate may contain solutions comprising antibody-nucleic acid conjugates with a nucleic acid including a second nucleic acid sequence, wherein the first nucleic acid sequence and the second nucleic acid sequence are distinct. In a non-limiting example, a first well of a multi-well plate may contain antibody-nucleic acid conjugates including a nucleic acid including a first nucleic acid sequence, a second well of a multi-well plate may contain antibody-nucleic acid conjugates comprising a nucleic acid including the first nucleic acid sequence, and a third well of the multi-well plate may contain antibody-nucleic acid conjugates including a nucleic acid comprising a second nucleic acid sequence, wherein the first nucleic acid sequence and the second nucleic acid sequence are distinct. In another non-limiting example, a first well of a multi-well plate may contain antibody-nucleic acid conjugates including an nucleic acid including a first nucleic acid sequence and a second nucleic acid sequence, a second well of a multi-well plate may contain antibody-nucleic acid conjugates including an nucleic acid including the first nucleic acid sequence and the second nucleic acid sequence, and a third well of the multi-well plate may contain antibody-nucleic acid conjugates including an nucleic acid including the second nucleic acid sequence and a third nucleic acid sequence, wherein the first nucleic acid sequence, the second nucleic acid sequence and the third nucleic acid sequence are distinct.

In some aspects of the preceding methods, step (a) includes the solution and a purification solution comprising polyethylene glycol (PEG) and sodium chloride (NaCl) to produce a mixture including antibody-nucleic acid conjugates, unconjugated nucleic acid, PEG and NaCl such that the concentration of PEG in the mixture is between about 5% to about 30% (w/v), or such that the concentration of PEG in the mixture is between about 7.5% to about 27.5% (w/v), or between about 10% to about 25% (w/v), or between about 12.5% to about 22.5% (w/v), or between about 15% to about 20% (w/v), or between about 5% to about 25% (w/v), or between about 7.5% to about 25% (w/v), or between about 12.5% to about 25% (w/v), or between about 15% to about 25% (w/v), or between about 17.5% to about 25% (w/v), or between about 20% or about 25% (w/v), or between about 22.5% to about 25%, or between about 10% to about 22.5% (w/v), or between about 10% to about 20% (w/v), or between about 10% to about 17.5% (w/v), or between about 10% to about 15% (w/v), or between about 10% to about 12.5% (w/v). In some aspects, the concentration of PEG in the mixture can be any range within the ranges described above. In some aspects, the concentration of PEG in the mixture is about 10% to about 25% (w/v).

In some aspects of the preceding methods, step (a) includes contacting the solution and a purification solution including polyethylene glycol (PEG) and sodium chloride (NaCl) to produce a mixture including antibody-nucleic acid conjugates, unconjugated nucleic acid, PEG and NaCl such that the concentration of NaCl in the mixture is between about 150 mM to about 400 mM, or between about 175 mM to about 375 mM, or between about 200 mM to about 350 mM, or between about 225 mM to about 325 mM, or between about 250 mM to about 300 mM, or about between about 150 mM to about 350 mM, or between about 175 mM to about 350 mM, or between about 225 mM to about 350 mM, or between about 250 mM to about 350 mM, or between about 275 mM to about 350 mM, or between about 300 mM to about 350 mM, or between about 325 mM to about 350 mM, or between about 200 mM to about 325 mM, or between about 200 mM to about 300 mM, or between about 200 mM to about 275 mM, or between about 200 mM to about 250 mM, or between about 200 mM to about 250 mM, or between about 200 mM to about 225 mM, or about 100 mM to about 250 mM, or about 125 mM to about 225 mM, or about 150 mM to about 200 mM, or about 100 mM to about 225 mM, or about 100 mM to about 200 mM, or about 100 mM to about 175 mM, or about 100 mM to about 150 mM, or about 100 mM to about 125 mM, or about 125 mM to about 250 mM, or about 150 mM to about 250 mM, or about 175 mM to about 250 mM, or about 200 mM to about 250 mM, or about 225 mM to about 250 mM. In some aspects, the concentration of NaCl in the mixture can be any range within the ranges described above. In some aspects, the concentration of NaCl in the mixture is about 200 mM to about 350 mM. In some aspects, the concentration of NaCl in the mixture is about 100 mM to about 250 mM.

Accordingly, the present disclosure provides methods for purifying antibody-nucleic acid conjugates from a solution including the antibody-nucleic acid conjugates and unconjugated nucleic acids, the method including a) contacting the solution and a purification solution comprising polyethylene glycol (PEG) and sodium chloride (NaCl) to produce a mixture including antibody-nucleic acid conjugates, unconjugated nucleic acids, PEG and NaCl such that the concentration of PEG in the mixture is between about 10% to about 25% and the concentration of NaCl in the mixture is between about 100 mM to about 250 mM; b) centrifuging the mixture to produce a supernatant and a pellet; c) isolating the pellet from the supernatant; d) resuspending the pellet with a resuspension solution to produce a purified solution; e) repeating steps (a)-(d) at least once using the purified solution of step (d) to produce a purified solution.

Accordingly, the present disclosure provides methods for purifying antibody-nucleic acid conjugates from a solution including the antibody-nucleic acid conjugates and unconjugated nucleic acids, the method including a) contacting the solution and a purification solution comprising polyethylene glycol (PEG) and sodium chloride (NaCl) to produce a mixture including antibody-nucleic acid conjugates, unconjugated nucleic acids, PEG and NaCl such that the concentration of PEG in the mixture is between about 10% to about 25% and the concentration of NaCl in the mixture is between about 100 mM to about 250 mM; b) centrifuging the mixture to produce a supernatant and a pellet; c) isolating the pellet from the supernatant; d) resuspending the pellet with a resuspension solution to produce a purified solution.

In some aspects, a purification solution can comprise PEG at a concentration of about 30%, or about 31%, or about 32%, or about 33%, or about 34%, or about 35%, or about 36%, or about 37%, or about 38%, or about 39%, or about 40%, or about 41%, or about 42%, or about 43%, or about 44%, or about 45%, or about 46%, or about 47%, or about 48%, or about 49%, or about 50% (w/v). In some aspects, a purification solution can comprise PEG at a concentration of between about 30% to about 50%, or between about 35% to about 45%. In some aspects, a purification can comprise PEG at a concentration of about 40%.

In some aspects, the purification solution can comprise NaCl at a concentration of about 150 mM, or about 155 mM, or about 160 mM, or about 165 mM, or about 170 mM, or about 175 mM, or about 180 mM, or about 185 mM, or about 190 mM, or about 195 mM, or about 200 mM, or about 205 mM, or about 210 mM, or about 215 mM, or about 220 mM, or about 225 mM, or about 230 mM, or about 235 mM, or about 240 mM, or about 245 mM, or about 250 mM. In some aspects, a purification solution can comprise NaCl at a concentration of between about 150 mM to about 250 mM, or between about 175 mM to about 225 mM. In some aspects, a purification solution can comprise NaCl at a concentration of about 200 mM.

Accordingly, in some aspects, a purification solution can comprise PEG at a concentration of between about 30% to about 50% (w/v) and NaCl at a concentration of about 150 mM to about 250 mM. In some aspects, a purification solution can comprise PEG at a concentration of about 40% (w/v) and NaCl at a concentration of about 200 mM.

In some aspects of the preceding methods, step (b) can comprise centrifuging the mixture at a speed of at least about 1,000 RCF (xg), or at least about 5,000 RCF(xg), or at least about 10,000 RCF (xg), or at least about 50,000 RCF (xg), or at least about 100,000 RCF (xg).

In some aspects of the preceding methods, step b) can comprise centrifuging the mixture for at least about 30 seconds, for at least about 1 minute, for at least about 1.5 minutes, for at least about 2 minutes, for at least about 2.5 minutes, for at least about 3 minutes, for at least about 3.5 minutes, for at least about 4 minutes, for at least about 5 minutes, for at least about 5.5 minutes, for at least about 6 minutes, for at least about 6.5 minutes, for at least about 7 minutes, for at least about 8 minutes, for at least about 8.5 minutes, for at least about 9 minutes, for at least about 9.5 minutes, or for at least about 10 minutes.

Accordingly, in some aspects of the preceding methods, step (b) can comprise centrifuging the mixture at a speed of at least about 1,000 RCF (xg) for at least about 2 minutes.

In some aspects of the preceding methods, step b) can comprise centrifuging the mixture for about 30 seconds, for about 1 minute, for about 1.5 minutes, for about 2 minutes, for about 2.5 minutes, for about 3 minutes, for about 3.5 minutes, for about 4 minutes, for about 5 minutes, for about 5.5 minutes, for about 6 minutes, for about 6.5 minutes, for about 7 minutes, for about 8 minutes, for about 8.5 minutes, for about 9 minutes, for about 9.5 minutes, or for about 10 minutes.

Accordingly, in some aspects of the preceding methods, step (b) can comprise centrifuging the mixture at a speed of at least about 1,000 RCF (xg) for about 2 minutes.

As would be appreciated by the skilled artisan, centrifuging the mixture can comprise the use of centrifugation method and centrifugation device known in the art, including, but not limited to, low-speed centrifugation, high-speed centrifugation, ultracentrifugation, fixed angle rotor centrifugation, swinging bucket rotor centrifugation, horizontal rotor centrifugation, vertical rotor centrifugation, benchtop centrifuges, continuous flow centrifuges, gas centrifuges, microcentrifuges, refrigerated centrifuges, vacuum centrifuges or any combination thereof.

Accordingly, the present disclosure provides methods for purifying antibody-nucleic acid conjugates from a solution comprising the antibody-nucleic acid conjugates and unconjugated nucleic acids, the method comprising a) contacting the solution and a purification solution comprising polyethylene glycol (PEG) and sodium chloride (NaCl) to produce a mixture comprising antibody-nucleic acid conjugates, unconjugated nucleic acids, PEG and NaCl such that the concentration of PEG in the mixture is between about 10% to about 25% and the concentration of NaCl in the mixture is between about 200 mM to 350 mM; b) centrifuging the mixture at a speed of at least about 1,000 RCF to produce a supernatant and a pellet; c) isolating the pellet from the supernatant; d) resuspending the pellet with a resuspension solution to produce a purified solution; e) repeating steps (a)-(d) at least once using the purified solution of step (d) to produce a purified solution.

As would be appreciated by the skilled artisan, polyethylene glycol (PEG) is a polymer of ethylene oxide. PEG is available in a variety of different molecular weights based on the length of the polymer. Accordingly, in some aspects of the preceding methods, the PEG can be PEG-100, PEG-200, PEG-300, PEG-400, PEG-500, PEG-600, PEG-700, PEG-800, PEG-900, PEG-1000, PEG-1500, PEG-2000, PEG-3000, PEG-3350, PEG-4000, PEG-5000, PEG-6000, PEG-7000, PEG-8000, PEG-9000, PEG-10000, PEG-15000, PEG-20000 or PEG-35000. In some aspects, the PEG can be PEG-6000. In some aspects, the PEG can be PEG-8000.

Accordingly, the present disclosure provides methods for purifying antibody-nucleic acid conjugates from a solution including the antibody-nucleic acid conjugates and unconjugated nucleic acids, the method comprising a) contacting the solution and a purification solution including polyethylene glycol (PEG)-6000 and sodium chloride (NaCl) to produce a mixture including antibody-nucleic acid conjugates, unconjugated nucleic acids, PEG and NaCl such that the concentration of PEG in the mixture is between about 10% to about 25% and the concentration of NaCl in the mixture is between about 200 mM to 350 mM; b) centrifuging the mixture at a speed of at least about 1,000 RCF to produce a supernatant and a pellet; c) isolating the pellet from the supernatant; d) resuspending the pellet with a resuspension solution to produce a purified solution; e) repeating steps (a)-(d) at least once using the purified solution of step (d) to produce a purified solution.

Accordingly, the present disclosure provides methods for purifying antibody-nucleic acid conjugates from a solution comprising the antibody-nucleic acid conjugates and unconjugated nucleic acids, the method comprising a) contacting the solution and a purification solution comprising polyethylene glycol (PEG)-6000 and sodium chloride (NaCl) to produce a mixture comprising antibody-nucleic acid conjugates, unconjugated nucleic acids, PEG and NaCl such that the concentration of PEG in the mixture is between about 10% to about 25% and the concentration of NaCl in the mixture is between about 100 mM to about 250 mM; b) centrifuging the mixture at a speed of at least about 1,000 RCF to produce a supernatant and a pellet; c) isolating the pellet from the supernatant; d) resuspending the pellet with a resuspension solution to produce a purified solution.

Accordingly, the present disclosure provides methods for purifying antibody-nucleic acid conjugates from a solution including the antibody-nucleic acid conjugates and unconjugated nucleic acids, the method comprising a) contacting the solution and a purification solution including polyethylene glycol (PEG)-8000 and sodium chloride (NaCl) to produce a mixture including antibody-nucleic acid conjugates, unconjugated nucleic acids, PEG and NaCl such that the concentration of PEG in the mixture is between about 10% to about 25% and the concentration of NaCl in the mixture is between about 200 mM to 350 mM; b) centrifuging the mixture at a speed of at least about 1,000 RCF to produce a supernatant and a pellet; c) isolating the pellet from the supernatant; d) resuspending the pellet with a resuspension solution to produce a purified solution; e) repeating steps (a)-(d) at least once using the purified solution of step (d) to produce a purified solution.

Accordingly, the present disclosure provides methods for purifying antibody-nucleic acid conjugates from a solution comprising the antibody-nucleic acid conjugates and unconjugated nucleic acids, the method comprising a) contacting the solution and a purification solution comprising polyethylene glycol (PEG)-8000 and sodium chloride (NaCl) to produce a mixture comprising antibody-nucleic acid conjugates, unconjugated nucleic acids, PEG and NaCl such that the concentration of PEG in the mixture is between about 10% to about 25% and the concentration of NaCl in the mixture is between about 100 mM to about 250 mM; b) centrifuging the mixture at a speed of at least about 1,000 RCF to produce a supernatant and a pellet; c) isolating the pellet from the supernatant; d) resuspending the pellet with a resuspension solution to produce a purified solution.

In some aspects of the preceding methods, contacting the solution and a purification solution in step (a) can comprise contacting the solution and the purification solution at a ratio of about 0.1:1 (v/v), or about 0.5:1 (v/v), or about 1:1 (v/v), or about 1.5:1 (v/v), or about 2:1 (v/v), or about 3:1 (v/v), or about 4:1 (v/v), or about 5:1 (v/v), or about 10:1 (v/v), or about 1:0.1 (v/v), or about 1:0.5 (v), or about 1:1.5 (v/v), or about 1:2 (v/v), or about 1:3 (v/v), or about 1:4 (v/v), or about 1:5 (v/v), or about 1:10 (v/v). In some aspects of the preceding methods, contacting the solution and a purification solution in step (a) can comprise contacting the solution and the purification solution at a ratio of about 1:1 (v/v).

In some aspects of the preceding methods, the solution including antibody-nucleic acid conjugates and unconjugated nucleic acids can further comprise unconjugated antibodies. In some aspects of the preceding methods, the mixture including antibody-nucleic acid conjugates, unconjugated nucleic acids, PEG and NaCl can further comprise unconjugated antibodies.

In some aspects of the preceding methods, step (a) can further comprise vortexing and/or agitating the mixture after the solution and the purification solution are brought into contact. Without wishing to be bound by theory, by vortexing and/or agitating the mixture after the solution and the purification solution are brought into contact ensures that the mixture is sufficiently homogenized. Vortexing and/or agitating can be accomplished using any method known in the art and can be performed for at least about 1 second, or at least about 2 seconds, or at least about 3 seconds, or at least about 4 seconds, or at least about 5 seconds, or at least about 6 seconds, or at least about 7 seconds, or at least about 8 seconds, or at least about 9 seconds, or at least about 10 seconds, or for about 1 second, or about 2 seconds, or about 3 seconds, or about 4 seconds, or about 5 seconds, or about 6 seconds, or about 7 seconds, or about 8 seconds, or about 9 seconds, or about 10 seconds.

In some aspects of the preceding methods, step (a) can comprise incubating the mixture for between about 10 minutes to about 50 minutes, or between about 15 minutes to about 45 minutes, or between about 20 to about 40 minutes, or between about 25 to about 35 minutes, or between about 20 minutes to about 50 minutes, or between about 20 minutes to about 45 minutes, or between about 20 minutes to about 35 minutes, or between about 20 to about 30 minutes, or between about 20 to about 25 minutes, or between about 10 minutes to about 40 minutes, or between about 15 minutes to about 40 minutes, or between about 25 minutes to about 40 minutes, or between about 30 minutes to about 40 minutes, or between about 35 minutes to about 40 minutes.

In some aspects of the preceding methods, step (a) can comprise incubating the mixture for at least about 10 minutes, or at least about 15 minutes, or at least about 20 minutes, or at least about 20 minutes, or at least about 25 minutes, or at least about 30 minutes, or at least about 35 minutes, or at least about 40 minutes, or at least about 45 minutes, or at least about 50 minutes. In some aspects of the preceding methods, step (a) can comprise incubating the mixture for about 10 minutes, or about 15 minutes, or about 20 minutes, or about 20 minutes, or about 25 minutes, or about 30 minutes, or about 35 minutes, or about 40 minutes, or about 45 minutes, or about 50 minutes.

In some aspects of the preceding methods, step (a) can comprise incubating the mixture for any of the time periods described above at about room temperature (e.g. about 20° C.). In some aspects of the preceding methods, step (a) can comprise incubating the mixture for any of the time periods described above at a temperature of about 18° C. to about 22° C.

Accordingly, in some aspects of the preceding methods, step (a) can comprise incubating the mixture for between about 20 to about 40 minutes at about 20° C.

Accordingly, in some aspects of the preceding methods, step (a) can comprise incubating the mixture for about 20 minutes at about 20° C.

In some aspects of the preceding methods, isolating the pellet from the supernatant in step (c) can be accomplished using any method known in the art to isolate a pellet from a supernatant, including, but not limited to, manually removing the supernatant from the pellet using a pipette, capillary, microcapillary, micropipette or any combination thereof, or removing the supernatant from the pellet using an automated liquid handling device, wherein the automated liquid handling device aspirates the supernatant away from the pellet using a pipette, a capillary, a microcapillary, a micropipette or any combination thereof.

In some aspects of the preceding methods, step (d) can comprise incubating the resuspended pellet for between about 10 minutes to about 50 minutes, or between about 15 minutes to about 45 minutes, or between about 20 to about 40 minutes, or between about 25 to about 35 minutes, or between about 20 minutes to about 50 minutes, or between about 20 minutes to about 45 minutes, or between about 20 minutes to about 35 minutes, or between about 20 to about 30 minutes, or between about 20 to about 25 minutes, or between about 10 minutes to about 40 minutes, or between about 15 minutes to about 40 minutes, or between about 25 minutes to about 40 minutes, or between about 30 minutes to about 40 minutes, or between about 35 minutes to about 40 minutes.

In some aspects of the preceding methods, step (d) can comprise incubating the resuspended pellet for at least about 10 minutes, or at least about 15 minutes, or at least about 20 minutes, or at least about 20 minutes, or at least about 25 minutes, or at least about 30 minutes, or at least about 35 minutes, or at least about 40 minutes, or at least about 45minutes, or at least about 50 minutes. In some aspects of the preceding methods, step (d) can comprise incubating the resuspended pellet for about 10 minutes, or about 15 minutes, or about 20 minutes, or about 20 minutes, or about 25 minutes, or about 30 minutes, or about 35 minutes, or about 40 minutes, or about 45 minutes, or about 50 minutes.

In some aspects of the preceding methods, step (d) can comprise incubating the resuspended pellet at about 37° C. In some aspects of the preceding methods, step (d) can comprise incubating the resuspended pellet at about 35° C. to about 39° C.

Accordingly, in some aspects of the preceding methods, step (d) can comprise incubating the resuspended pellet for between about 20 to about 40 minutes at about 37° C. Accordingly, in some aspects of the preceding methods, step (d) can comprise incubating the resuspended pellet for about 20 minutes at about 20° C.

In some aspects of the preceding methods, the resuspension solution can comprise any solution known in the art that is compatible with the antibody-nucleic acid conjugate. In a non-limiting example, the resuspension solution can comprise phosphate buffered saline. In a non-limiting example, the resuspension solution can comprise at least one biological buffer (e.g. at least one of HEPES, MOPS, MES, BES, MOPSO, ACES, TAPS, Bicine, Tricine or any other biological buffer known in the art). In some aspects, the resuspension solution can comprise at least one salt.

In some aspects of the preceding methods, steps (a)-(d) can be repeated at least once, or at least twice, or at least three times, or at least four times, or at least five times, or at least six times, or at least seven times, or at least eight times, or at least nine times, or at least ten times.

In some aspects of the preceding methods, the purified solution after step (d) or after repeating steps (a)-(d) at least once can be at least about 90%, or at least about 91%, or at least about 92%, or at least about 93%, or at least about 94%, or at least about 95%, or at least about 96%, or at least about 97%, or at least about 98%, or at least about 99%, or at least about 99.5% pure for the antibody-nucleic acid conjugates, as measured by size exclusion (SEC) high performance liquid chromatography (HPLC).

In some aspects of the preceding methods, the purified solution after step (d) or after repeating steps (a)-(d) at least once can be about 90%, or about 91%, or about 92%, or about 93%, or about 94%, or about 95%, or about 96%, or about 97%, or about 98%, or about 99%, or about 99.5% pure for the antibody-nucleic acid conjugates, as measured by size exclusion (SEC) high performance liquid chromatography (HPLC).

In some aspects of the preceding methods, the purified solution after step (d) or after repeating steps (a)-(d) at least one can be between about 85% to about 95%, or between about 87.5% to about 97.5%, or between about 90 to about 99%, or between about 90% to about 95% pure for the antibody-nucleic acid conjugates, as measured by size exclusion (SEC) high performance liquid chromatography (HPLC).

In some aspects of the preceding methods, the purified solution after repeating steps (a)-(d) at least once can be at least about 90%, or at least about 91%, or at least about 92%, or at least about 93%, or at least about 94%, or at least about 95%, or at least about 96%, or at least about 97%, or at least about 98%, or at least about 99%, or at least about 99.5% pure for the antibody-nucleic acid conjugates, as measured by size exclusion (SEC) high performance liquid chromatography (HPLC).

In some aspects, purity as measured by SEC HPLC is calculated by determining the area under the 260 nm curve that corresponds to the antibody-nucleic acid conjugate and dividing it by the sum of the area under the 260 nm curve that corresponds to the antibody-nucleic acid conjugate and the area under the 260 nm curve that corresponds to the free, unconjugated nucleic acid. That is, purity can be calculated using the following formula:

$$\% Purity = \frac{( \begin{matrix}Area under 260 nm curve for antibody - \\ nucleic acid conjugate peak\end{matrix} )}{( \begin{matrix}\begin{matrix}\begin{matrix}Area under 260 nm curve for antibody - \\ nucleic acid conjugate peak +\end{matrix} \\ Area under 260 nm curve for the free ,\end{matrix} \\ unconjugated nucleic acid\end{matrix} )} \times 100 \%$$

In some aspects of the preceding methods, the yield of the antibody-nucleic acid conjugates is at least about 60%, or at least about 62.5%, or at least about 65%, or at least about 67.5%, or at least about 70%, or at least about 72.5%, or at least about 75%, or at least about 77.5%, or at least about 80%, or at least about 82.5%, or at least about 85%, or at least about 87.5%, or at least about 90%, or at least about 92.5%, or at least about 95%, or at least about 97.5%, or at least about 99%, or at least about 99.5%. In some aspects of the preceding methods, the yield of the antibody-nucleic acid conjugates is 60%, or 62.5%, or 65%, or 67.5%, or 70%, or 72.5%, or 75%, or 77.5%, or 80%, or 82.5%, or 85%, or 87.5%, or 90%, or 92.5%, or 95%, or 97.5%, or 99%, or 99.5%. In some aspects of the preceding methods, the yield of the antibody-nucleic acid conjugates is at least about 80%.

As would be appreciated by the skilled artisan the yield can be calculated by measuring the amount of antibody-nucleic acid conjugate prior to and after purification, and then applying the following formula:

$$\% Yield = \frac{\begin{matrix}Amount of antibody - \\ nucleic acid conjugate after purification\end{matrix}}{\begin{matrix}Amount of antibody - \\ nucleic acid conjugate prior to purification\end{matrix}} \times 100 \%$$

The amount of antibody-nucleic acid conjugate can be measured using any method known in the art to quantify the amount of an antibody-nucleic acid conjugate.

As would be appreciated by the skilled artisan, the yield by measuring the amount of antibody (i.e. amount of protein) prior to and after purification, and then applying the following formula:

$$\% Yield = \frac{Amount of antibody after purification}{Amount of antibody prior to purification} \times 100 \%$$

In embodiments wherein the purification of an antibody-nucleic acid conjugate is preceded by a conjugation reaction, the yield can be calculated by measuring the amount of antibody (i.e. amount of protein) that was input into the conjugation reaction and the amount of antibody (i.e. amount of protein) obtained after purification, and then applying the following formula:

$$\% Yield = \frac{Amount of antibody after purification}{Amount of antibody added to conjugation reaction} \times 100 \%$$

The amount of antibody can be measured using any method known in the art to quantify protein amounts, including, but not limited to Bradford protein assay, ultraviolet-visible spectroscopy, Biuret protein assay, Lowry protein assay, BCA protein assay, Amido black protein assay and Colloidal gold protein assay.

### Methods of Producing Antibody-nucleic Acid Conjugates

The present disclosure provides methods of producing antibody-nucleic acid conjugates comprising: i) contacting a first solution comprising nucleic acids including at least one reactive group and a second solution including antibodies under conditions such that the at least one reactive group of the nucleic acids reacts with the antibodies, thereby forming antibody-nucleic acid conjugates; ii) purifying the antibody-nucleic acid conjugates using any of the purification methods described herein. In some aspects of the preceding method, a buffer exchange step can be performed on one or more of the solutions prior to step (i) or following step (i) and prior to step (ii), using any buffer exchange procedure known in the art (e.g. spin filter buffer exchange, PD-10 columns, etc.). The skilled artisan would appreciate and readily ascertain the conditions under which the at least one reactive group of the nucleic acids reacts with the antibodies to form antibody-nucleic acid conjugates.

In some aspects of the preceding method, a reactive group can be any reactive group known in the art to be useful for covalently linking a nucleic acid molecule and a protein molecule (e.g. antibody). Non-limiting examples of reactive groups include, but are not limited to, maleimide, NHS esters, azides, alkynes, DBCOs, and tetrazines. Accordingly, an at least one reactive group comprises at least one moiety selected from the group consisting of a maleimide moiety, a iodoacetamide moiety, a benzylic halide moiety, a bromomethylketone moiety, an isothiocyanate moiety, a succinimidyl esters moiety, a carboxylic ester moiety, a sulfosuccinimidyl ester moiety, a 4-sulfotetrafluorophenyl (STP) ester moiety, a 2,4,5,6-Tetrafluorophenyl (TFP) ester moiety, a sulfodicholorphenol (SDP) ester moiety, a carbonyl azide moiety, a N-Hydroxysuccinimide (NHS) moiety, an azide moiety, an alkyne moiety, a tetrazine moiety, a aza-dibenzocyclooctyne moiety, and a sulfonyl chloride moiety.

As would be appreciated by the skilled artisan, an antibody-nucleic acid conjugate can be formed through a labeling reaction wherein a nucleic acid comprising one or more reactive groups reacts and forms a covalent bound with one or more amino acid side chains and/or portions of the polypeptide backbone. These amino acid side chains can include, but are not limited to, cysteine side chains, lysine side chains, or any other reactive side chain known in the art.

In aspects wherein an antibody-nucleic acid conjugate is to be formed through a cysteine labeling reaction (e.g. the nucleic acid comprises a maleimide reactive group), prior to step (i), the antibody can be treated with a reducing agent (e.g. TCEP, DTT or any other reducing agent known in the art).

In some aspects of the methods of producing antibody-nucleic acid conjugates described herein, the conjugation step can be performed in a multi-well plate, wherein each well comprises the same or different antibodies and/or nucleic acids. Subsequently, and prior to purification using the methods described herein, the antibody-nucleic acid conjugates produced in the different wells can be combined and purified together to produce a plurality of antibody-nucleic acid conjugates comprising a plurality of different species of antibody-nucleic acid conjugates.

### Antibody-nucleic Acid Conjugates of the Present Disclosure

As used herein, the term “antibody-nucleic acid conjugate” is used in the broadest sense to refer to a molecule that comprises one or more antibodies (described in further detail herein) that are covalently linked to one or more nucleic acids (described in further detail herein). In a non-limiting example, an antibody-nucleic acid conjugate can comprise a single antibody that is covalently linked to a single nucleic acid.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); single domain antibodies (sdAb); camelid VHH domains; and multispecific antibodies formed from antibody fragments.

As would be appreciated by the skilled artisan, an antibody can be specifically designed and/or modified such that it reacts with a nucleic acid comprising a reactive group at one or more specific sites on the antibody. In a non-limiting example, antibodies can be designed to have a single exposed cysteine that can be reacted with a nucleic acid comprising a maleimide group in order to form an antibody-nucleic acid conjugate.

### Nucleic Acids of the Present Disclosure

As used herein, the term “nucleic acid” is used in the broadest sense to refer to a molecule comprising one or more polynucleotide chains.

As used herein, the term “polynucleotide” is used in the broadest sense to describe a polymer comprising two or more nucleotide monomers that are covalently bonded in a chain.

Nucleic acids of the present disclosure can be single-stranded (i.e. composed of a single polynucleotide chain), double-stranded (i.e. composed of two polynucleotide chains hybridized together through Watson-Crick or non-Watson base pairing), or partially double-stranded (i.e. having both single-stranded and double-stranded regions). Nucleic acids of the present disclosure can comprise any number of polynucleotide chains. Polynucleotide chains that are hybridized together can be “perfectly matched”, such that the polynucleotide chains form a double stranded structure with one another such that every nucleotide in each strand undergoes Watson-Crick base pairing with a nucleotide in the other strand. Polynucleotide chains that are hybridized can comprise at least one mismatch, wherein the term “mismatch” means that a pair of nucleotides in the duplex fail to undergo Watson-Crick bonding.

Nucleic acids of the present disclosure can comprise at least one natural base. Nucleic acids of the present disclosure can comprise no natural bases. Nucleic acids of the present disclosure can comprise at least one modified nucleotide or nucleic acid analog. Nucleic acids of the present disclosure can comprise no modified nucleotides or nucleic acid analogs. Nucleic acids of the present disclosure can comprise at least one universal base. Nucleic acids of the present disclosure can comprise no universal bases.

Nucleic acids of the present disclosure can comprise any combination natural bases (e.g. 0, 1, 2, 3, 4, 5, 6, 7, 8, 9, 10, or more natural bases), modified nucleotides or nucleic acid analogs (e.g. 0, 1, 2, 3, 4, 5, 6, 7, 8, 9, 10, or more modified nucleotides or nucleic acid analogs), and/or universal bases (e.g. 0, 1, 2, 3, 4, 5, 6, 7, 8, 9, 10, or more universal bases). When present in a combination, the natural bases, modified nucleotides or nucleic acid analogs, and/or universal bases of a nucleic acid can be arranged in any order.

The terms “modified nucleotides” or “nucleic acid analogues” include, but are not limited to, locked nucleic acids (LNA), bridged nucleic acids (BNA), propyne-modified nucleic acids, zip nucleic acids (ZNA®), isoguanine, isocytosine 6-amino-1-(4-hydroxy-5-hydroxy methyl-tetrahydro-furan-2-yl)-1,5-dihydro-pyrazolo [3,4-d] pyrimidin-4-one (PPG) and 2′-modified nucleic acids such as 2′-O-methyl nucleic acids. The target binding domain can include zero to six (e.g. 0, 1, 2, 3, 4, 5 or 6) modified nucleotides or nucleic acid analogues. Preferably, the modified nucleotides or nucleic acid analogues are locked nucleic acids (LNAs).

The term “locked nucleic acids (LNA)” as used herein includes, but is not limited to, a modified RNA nucleotide in which the ribose moiety comprises a methylene bridge connecting the 2′ oxygen and the 4′ carbon. This methylene bridge locks the ribose in the 3′-endo confirmation, also known as the north confirmation, that is found in A-form RNA duplexes. The term inaccessible RNA can be used interchangeably with LNA. The term “bridged nucleic acids (BNA)” as used herein includes, but is not limited to, modified RNA molecules that comprise a five-membered or six-membered bridged structure with a fixed 3′-endo confirmation, also known as the north confirmation. The bridged structure connects the 2′ oxygen of the ribose to the 4′ carbon of the ribose. Various different bridge structures are possible containing carbon, nitrogen, and hydrogen atoms. The term “propyne-modified nucleic acids” as used herein includes, but is not limited to, pyrimidines, namely cytosine and thymine/uracil, that comprise a propyne modification at the C5 position of the nucleic acid base. The term “zip nucleic acids (ZNA®)” as used herein includes, but is not limited to, polynucleotides that are conjugated with cationic spermine moieties.

The term “universal base” as used herein includes, but is not limited to, a nucleotide base does not follow Watson-Crick base pair rules but rather can bind to any of the four canonical bases (A, T/U, C, G) located on the target nucleic acid.

In some aspects, a nucleic acid of the present disclosure can comprise at least one cleavable moiety. Exemplary cleavable moieties include, but are not limited to, chemically cleavable moiety (e.g. a moiety that is cleaved when exposed to a particular chemical, combination of chemicals or reaction conditions), a photo-cleavable moiety (e.g. a moiety that is cleaved when exposed to light of a sufficient wavelength or light comprising a sufficient range of wavelengths), or an enzymatically cleavable moiety (e.g. a moiety that is cleaved by a specific enzyme or class of enzymes).

In some aspects, a nucleic acid of the present disclosure can comprise, consist essentially of, or consist of deoxyribonucleic acid (DNA). In some aspects, a nucleic acid of the present disclosure can comprise, consist essentially of, or consist of ribonucleic acid (RNA). In some aspects a nucleic acid of the present disclosure can comprise, consist essentially of, or consist of a combination of DNA and RNA.

In some aspects, the DNA can be D-DNA (right-turning). Accordingly, in some aspects, a nucleic acid of the present disclosure can comprise, consist essentially of, or consist of D-DNA.

In some aspects, the DNA can be L-DNA (left turning). Accordingly, in some aspects, a nucleic acid of the present disclosure can comprise, consist essentially of, or consist of L-DNA.

In some aspects, a nucleic acid of the present disclosure can be at least about one, or at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40, or at least about 41, or at least about 42, or at least about 43, or at least about 44, or at least about 45, or at least about 46, or at least about 47, or at least about 48, or at least about 49, or at least about 50, or at least about 51, or at least about 52, or at least about 53, or at least about 54, or at least about 55, or at least about 56, or at least about 57, or at least about 58, or at least about 59, or at least about 60, or at least about 61, or at least about 62, or at least about 63, or at least about 64, or at least about 65, or at least about 66, or at least about 67, or at least about 68, or at least about 69, or at least about 70, or at least about 71, or at least about 72, or at least about 73, or at least about 74, or at least about 75, or at least about 76, or at least about 77, or at least about 78, or at least about 79, or at least about 80, or at least about 81, or at least about 82, or at least about 83, or at least about 84, or at least about 85, or at least about 86, or at least about 87, or at least about 88, or at least about 89, or at least about 90, or at least about 91, or at least about 92, or at least about 93, or at least about 94, or at least about 95, or at least about 96, or at least about 97, or at least about 98, or at least about 99, or at least about 100 nucleotides in length.

In some aspects, a nucleic acid of the present disclosure can be about one, or about two, or about three, or about four, or about five, or about six, or about seven, or about eight, or about nine, or about ten, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 33, or about 34, or about 35, or about 36, or about 37, or about 38, or about 39, or about 40, or about 41, or about 42, or about 43, or about 44, or about 45, or about 46, or about 47, or about 48, or about 49, or about 50, or about 51, or about 52, or about 53, or about 54, or about 55, or about 56, or about 57, or about 58, or about 59, or about 60, or about 61, or about 62, or about 63, or about 64, or about 65, or about 66, or about 67, or about 68, or about 69, or about 70, or about 71, or about 72, or about 73, or about 74, or about 75, or about 76, or about 77, or about 78, or about 79, or about 80, or about 81, or about 82, or about 83, or about 84, or about 85, or about 86, or about 87, or about 88, or about 89, or about 90, or about 91, or about 92, or about 93, or about 94, or about 95, or about 96, or about 97, or about 98, or about 99, or about 100 nucleotides in length.

In some aspects, a nucleic acid of the present disclosure can be more than about 100 nucleotides in length.

In some aspects, a nucleic acid of the present disclosure can be about 60 to about 70 nucleotides in length. In some aspects, a nucleic acid of the present disclosure can be about 66 nucleotides in length.

In some aspects, a nucleic acid of the present disclosure can be about 50 to about 60 nucleotides in length. In some aspects, a nucleic acid of the present disclosure can be about 56 nucleotides in length.

### Kits of the Present Disclosure

The present disclosure provides kits for use in the methods described herein.

The present disclosure provides a kit comprising a purification solution comprising PEG and NaCl.

In some aspects, a purification solution can comprise PEG at a concentration of about 30%, or about 31%, or about 32%, or about 33%, or about 34%, or about 35%, or about 36%, or about 37%, or about 38%, or about 39%, or about 40%, or about 41%, or about 42%, or about 43%, or about 44%, or about 45%, or about 46%, or about 47%, or about 48%, or about 49%, or about 50% (w/v). In some aspects, a purification solution can comprise PEG at a concentration of between about 30% to about 50%, or between about 35% to about 45%. In some aspects, a purification can comprise PEG at a concentration of about 40%.

As would be appreciated by the skilled artisan, polyethylene glycol (PEG) is a polymer of ethylene oxide. PEG is available in a variety of different molecular weights based on the length of the polymer. Accordingly, in some aspects of the preceding methods, the PEG can be PEG-100, PEG-200, PEG-300, PEG-400, PEG-500, PEG-600, PEG-700, PEG-800, PEG-900, PEG-1000, PEG-1500, PEG-2000, PEG-3000, PEG-3350, PEG-4000, PEG-5000, PEG-6000, PEG-7000, PEG-8000, PEG-9000, PEG-10000, PEG-15000, PEG-20000 or PEG-35000. In some aspects, the PEG can be PEG-6000. In some aspects, the PEG can be PEG-8000.

In some aspects, the purification solution can comprise NaCl at a concentration of about 150 mM, or about 155 mM, or about 160 mM, or about 165 mM, or about 170 mM, or about 175 mM, or about 180 mM, or about 185 mM, or about 190 mM, or about 195 mM, or about 200 mM, or about 205 mM, or about 210 mM, or about 215 mM, or about 220 mM, or about 225 mM, or about 230 mM, or about 235 mM, or about 240 mM, or about 245 mM, or about 250 mM. In some aspects, a purification solution can comprise NaCl at a concentration of between about 150 mM to about 250 mM, or between about 175 mM to about 225 mM. In some aspects, a purification solution can comprise NaCl at a concentration of about 200 mM.

Accordingly, in some aspects, a purification solution can comprise PEG at a concentration of between about 30% to about 50% (w/v) and NaCl at a concentration of about 150 mM to about 250 mM. In some aspects, a purification solution can comprise PEG at a concentration of about 40% (w/v) and NaCl at a concentration of about 200 mM.

In some aspects, a kit can further comprise at least one solution comprising nucleic acids of the present disclosure, wherein the nucleic acids comprise at least one reactive group. A reactive group can be any reactive group known in the art to be useful for covalently linking a nucleic acid molecule and a protein molecule (e.g. antibody). Non-limiting examples of reactive groups include, but are not limited to, maleimide, NHS esters, azides, alkynes, DBCOs, and tetrazines.

In some aspects, a kit can further comprise a first solution comprising nucleic acids of the present disclosure, wherein the nucleic acids comprise at least one reactive group and an at least second solution comprising nucleic acids of the present disclosure, wherein the nucleic acids comprise at least one reactive group, wherein the nucleic acids in the first solution comprise a nucleic acid sequence that is wholly or at least partially different from the nucleic acid sequence of the nucleic acids in the at least second solution.

In some aspects, a kit can further comprise a solution comprising at least one reducing agent (e.g. TCEP, DTT or any other reducing agent known in the art).

In some aspects, a kit can further comprise a solution comprising antibodies of the present disclosure.

In some aspects, a kit can further comprise at least one spin filter.

In some aspects, a kit can further comprise instructions for using the kit to perform at least one method described herein. In some aspects, the instructions can be written instructions.

### Exemplary Embodiments

Embodiment 1. A method for purifying antibody-nucleic acid conjugates from a solution including the antibody-nucleic acid conjugates and unconjugated nucleic acid, the method comprising:
a) contacting the solution and a purification solution including polyethylene glycol (PEG) and sodium chloride (NaCl) to produce a mixture including antibody-nucleic acid conjugates, unconjugated nucleic acids, PEG and NaCl such that the concentration of PEG in the mixture is between about 5% to about 30% (w/v) and the concentration of NaCl in the mixture is between about 100 mM to about 250 mM;
b) centrifuging the mixture to produce a supernatant and a pellet;
c) isolating the pellet from the supernatant;
d) resuspending the pellet with a resuspension solution to produce a purified solution;
e) repeating steps (a)-(d) at least once using the purified solution of step (d) to produce a purified solution that is at least 90% pure for the antibody-nucleic acid conjugates, as measured by size exclusion (SEC) high performance liquid chromatography (HPLC).

Embodiment 2. The method of embodiment 1, wherein the concentration of PEG in the mixture is between about 10% to about 25% (w/v).

Embodiment 3. The method of embodiment 1 or embodiment 2, wherein the concentration of NaCl in the mixture is between about 150 mM to about 200 mM.

Embodiment 4. The method of any one of the preceding embodiments, wherein the purified solution is at least 95% pure for the antibody-nucleic acid conjugates, as measured by size exclusion (SEC) high performance liquid chromatography (HPLC).

Embodiment 5. The method of any one of the preceding embodiments, wherein step (b) comprises centrifuging the mixture at a speed of at least about 1,000 RCF (xg).

Embodiment 6. The method of any one of the preceding embodiments, wherein the centrifuging in step (b) is performed for at least about 2 minutes.

Embodiment 7. The method of any one of the preceding embodiments, wherein step (a) further comprises incubating the mixture for about 20 to about 40 minutes at about 20° C.

Embodiment 8. The method of any one of the preceding embodiments, wherein step (d) further comprises incubating the resuspended pellet for about 20 to about 40 minutes at about 37° C.

Embodiment 9. The method of any one of the preceding embodiments, wherein step (a) further comprises vortexing the mixture.

Embodiment 10. The method of any one of the preceding embodiments, wherein the resuspension solution comprises phosphate buffered saline (PBS).

Embodiment 11. The method of any one of the preceding embodiments, wherein the PEG is:
i) PEG-6000;
ii) PEG-8000;
iii) PEG-10000; or
iv) PEG-35,000.

Embodiment 12. The method of embodiment 11, wherein the PEG is PEG-6000.

Embodiment 13. The method of embodiment 11, wherein the PEG is PEG-8000.

Embodiment 14. The method of any one of the preceding embodiments, wherein contacting the solution and a purification solution in step (a) comprises contacting the solution and the purification solution at a ratio of 1:1 (v/v).

Embodiment 15. The method of any one of the preceding embodiments, wherein the concentration of PEG in the purification solution is between about 35% to about 45% (w/v), preferably wherein the concentration of PEG in the purification solution is about 40% (w/v).

Embodiment 16. The method of any one of the preceding embodiments, wherein the concentration of NaCl in the purification solution is between about 150 mM to 250 mM, preferably wherein the concentration of NaCl in the purification solution is about 200 mM.

Embodiment 17. The method of any one of the preceding embodiments, wherein the antibodies comprise a Fab, a Fab′, a F(ab′)2, a Fv fragment, a single-chain (sc)Fv (scFv) antibody fragment, a linear antibody, a single domain antibody (sdAb), a camelid VHH domain, a multi-specific antibody or any combination thereof.

Embodiment 18. The method of any one of the preceding embodiments, wherein the nucleic acids are single-stranded, double-stranded or partially double-stranded.

Embodiment 19. The method of any one of the preceding embodiments, wherein the nucleic acids comprise L-DNA.

Embodiment 20. The method of any one of the preceding embodiments, wherein the method is performed in one or more wells of a multi-well plate, thereby simultaneously purifying a plurality of antibody-nucleic acid conjugates.

Embodiment 21. The method of any one of the preceding embodiments, wherein the method is performed in two or more wells of a multi-well plate, thereby simultaneously purifying a plurality of antibody-nucleic acid conjugates in distinct wells.

Embodiment 22. A method of producing antibody-nucleic acid conjugates, the method comprising:
a) contacting a first solution comprising nucleic acids including at least one reactive group and a second solution including antibodies under conditions such that the at least one reactive group of the nucleic acids reacts with the antibodies, thereby forming antibody-nucleic acid conjugates;
b) purifying the antibody nucleic acid conjugates using the method of any one of embodiments 1-21.

Embodiment 23. The method of embodiment 22, wherein the at least one reactive group comprises a maleimide moiety.

Embodiment 24. The method of embodiment 22 or embodiment 23, wherein the at least one reactive group comprises at least one moiety selected from the group consisting of a maleimide moiety, a iodoacetamide moiety, a benzylic halide moiety, a bromomethylketone moiety, an isothiocyanate moiety, a succinimidyl esters moiety, a carboxylic ester moiety, a sulfosuccinimidyl ester moiety, a 4-sulfotetrafluorophenyl (STP) ester moiety, a 2,4,5,6-Tetrafluorophenyl (TFP) ester moiety, a sulfodicholorphenol (SDP) ester moiety, a carbonyl azide moiety, a N-Hydroxysuccinimide (NHS) moiety, an azide moiety, an alkyne moiety, a tetrazine moiety, a aza-dibenzocyclooctyne moiety, and a sulfonyl chloride moiety.

Embodiment 25. A kit comprising:
i) a first solution including nucleic acids including at least one reactive group; and
ii) a purification solution including polyethylene glycol (PEG) and sodium chloride (NaCl).

Embodiment 26. The kit of embodiment 25, wherein the nucleic acids are single-stranded, double-stranded or partially double-stranded.

Embodiment 27. The kit of any one of embodiments 25-26, wherein the nucleic acids comprise L-DNA.

Embodiment 28. The kit of any one of embodiments 25-27, wherein the at least one reactive group comprises a maleimide moiety.

Embodiment 29. The method of any one of embodiments 25-27, wherein the at least one reactive group comprises at least one moiety selected from the group consisting of a maleimide moiety, a iodoacetamide moiety, a benzylic halide moiety, a bromomethylketone moiety, an isothiocyanate moiety, a succinimidyl esters moiety, a carboxylic ester moiety, a sulfosuccinimidyl ester moiety, a 4-sulfotetrafluorophenyl (STP) ester moiety, a 2,4,5,6-Tetrafluorophenyl (TFP) ester moiety, a sulfodicholorphenol (SDP) ester moiety, a carbonyl azide moiety, a N-Hydroxysuccinimide (NHS) moiety, an azide moiety, an alkyne moiety, a tetrazine moiety, a aza-dibenzocyclooctyne moiety, and a sulfonyl chloride moiety.

Embodiment 30. The kit of any one of embodiments 25-29, further comprising a second solution including antibodies.

Embodiment 31. The kit of embodiment 30, wherein the antibodies comprise a Fab, a Fab′, a F(ab′)2, a Fv fragment, a single-chain (sc)Fv (scFv) antibody fragment, a linear antibody, a single domain antibody (sdAb), a camelid VHH domain, a multi-specific antibody or any combination thereof.

Embodiment 32. The kit of any one of embodiments 25-31, further comprising a third solution including nucleic acids including at least one reactive group, wherein the nucleic acids in the first solution include a nucleic acid sequence that is different from the nucleic acid sequence of the nucleic acids in the third solution.

Embodiment 33. The kit of any one of embodiments 25-32, wherein the PEG is:
i) PEG-6000;
ii) PEG-8000;
iii) PEG-10000; or
iv) PEG-35,000.

Embodiment 34. The kit of embodiment 33, wherein the PEG is PEG-6000.

Embodiment 35. The kit of embodiment 33, wherein the PEG is PEG-8000.

Embodiment 36. The kit of any one of embodiments 25-35, wherein the concentration of PEG in the purification solution is between about 35% to about 50% (w/v), preferably wherein the concentration of PEG in the purification solution is about 40% (w/v).

Embodiment 37. The kit of any one of embodiments 25-36, wherein the concentration of NaCl in the purification solution is between about 150 mM to 250 mM, preferably wherein the concentration of NaCl in the purification solution is about 200 mM.

Embodiment 38. The kit of any one of embodiments 25-37, further comprising at least one aliquot of TCEP.

Embodiment 39. The kit of any one of embodiments 25-38, further comprising at least one spin filter.

Embodiment 40. The kit of any one of embodiments 25-39, further comprising at least one multi-well plate.

### EXAMPLES

### Example 1—Purification of Antibody-Nucleic Acid Conjugates Using the Methods of the Present Disclosure

The following is a non-limiting example that demonstrates that the purification methods of the present disclosure can be used to purify antibody-nucleic acid conjugates that are more than 90% pure from unconjugated nucleic acids left over from the conjugation reaction.

The antibody-nucleic acid conjugates were prepared by using standard cysteine-labeling methods known in the art. Briefly, antibodies were treated with the reducing agent TCEP, washed using a spin filter, and incubated with nucleic acids that included a reactive group capable of reacting with cysteine residues on the antibodies. These antibody-nucleic acid conjugates were then purified using one of two separate protocols.

In the first protocol, the conjugation reaction comprising the antibody-nucleic acid conjugates and the unconjugated nucleic acids was contacted with a purification solution comprising PEG-6000 to produce a mixture comprising antibody-nucleic acid conjugates, unconjugated nucleic acids and PEG such that the concentration of PEG in the mixture was about 25% (w/v). The mixture was then centrifuged to produce a supernatant and a pellet, the pellet was isolated from the supernatant, and the pellet was resuspended using a resuspension solution comprising PBS.

In the second protocol, the conjugation reaction comprising the antibody-nucleic acid conjugates and the unconjugated nucleic acids was contacted with a purification solution comprising PEG-6000 and NaCL to produce a mixture comprising antibody-nucleic acid conjugates, unconjugated nucleic acids, PEG and NaCl such that the concentration of PEG in the mixture was about 25% (w/v) and the concentration of NaCl was about 100 mM. The mixture was then centrifuged to produce a supernatant and a pellet, the pellet was isolated from the supernatant, and the pellet was resuspended using a resuspension solution comprising PBS.

The purified solutions were then analyzed using SEC HLPC to determine the extent of antibody-nucleic acid conjugation purification.

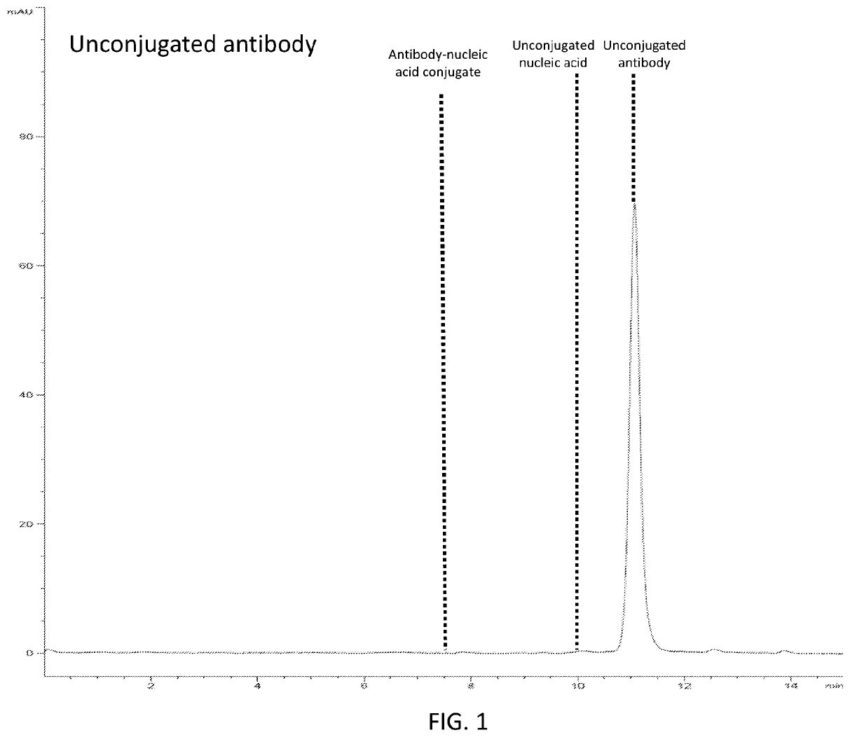

As a first control, unconjugated antibody was analyzed using SEC HLPC. The results of this analysis are shown in FIG. 1. The horizontal lines in FIG. 1, from left to right, denote the expected elution times of the antibody-nucleic acid conjugate, the unconjugated nucleic acid and the unconjugated antibody.

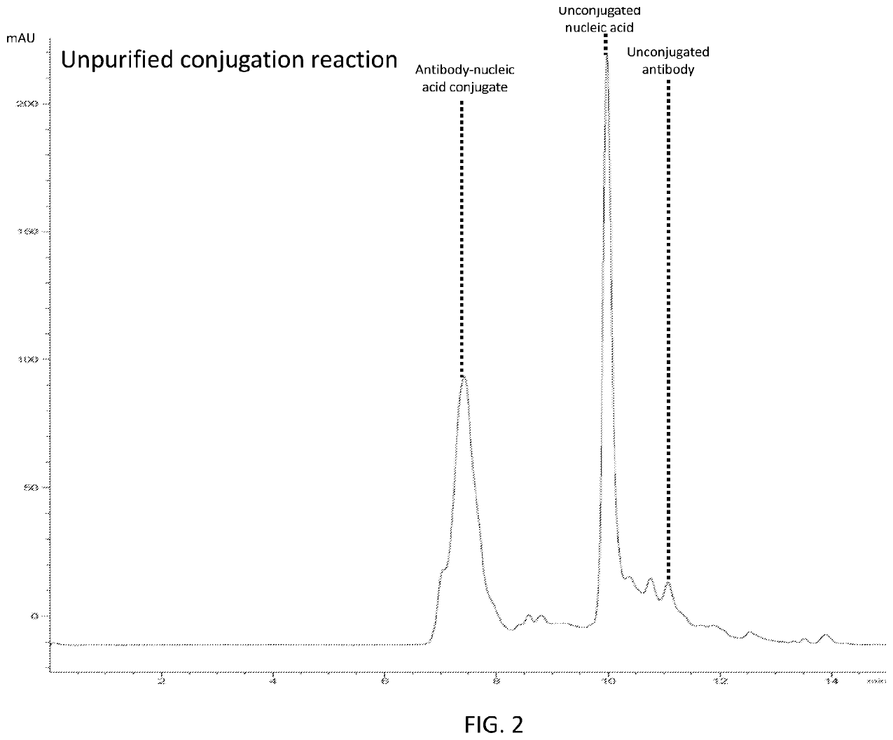

As a second control, an unpurified conjugation reaction was analyzed using SEC HLPC. The results of this analysis are shown in FIG. 2. The horizontal lines in FIG. 2, from left to right, denote the expected elution times of the antibody-nucleic acid conjugate, the unconjugated nucleic acid and the unconjugated antibody. As expected, the unpurified conjugation reaction shows an excess of unconjugated nucleic acid.

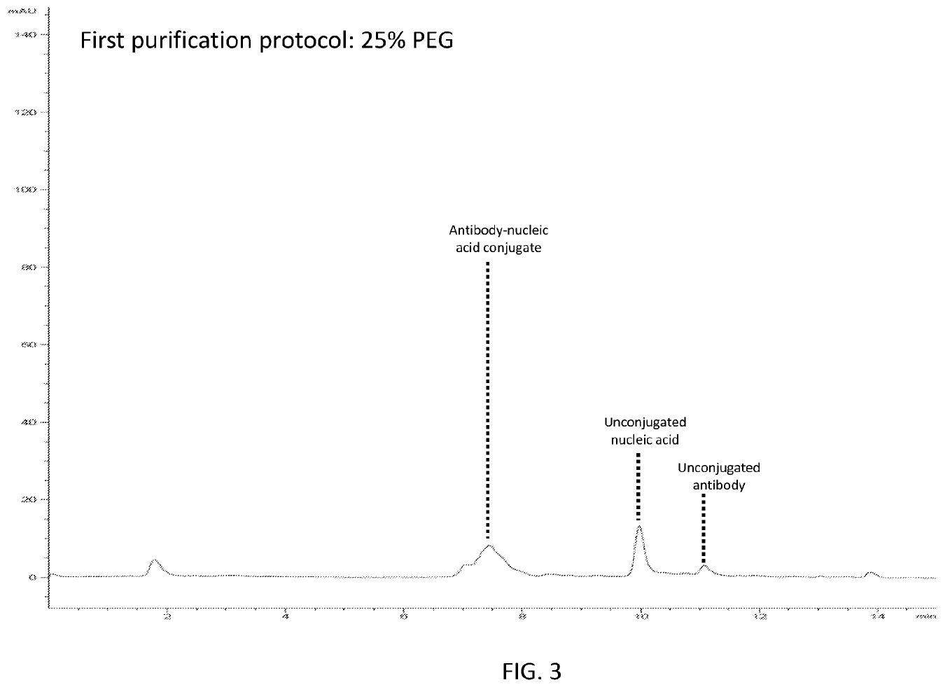

A conjugation reaction that was purified using the first purification protocol described above was analyzed using SEC HPLC. The results of this analysis are shown in FIG. 3. The horizontal lines in FIG. 3, from left to right, denote the expected elution times of the antibody-nucleic acid conjugate, the unconjugated nucleic acid and the unconjugated antibody. As shown in FIG. 3, the first purification protocol resulted in little to no purification of the antibody-nucleic acid conjugate.

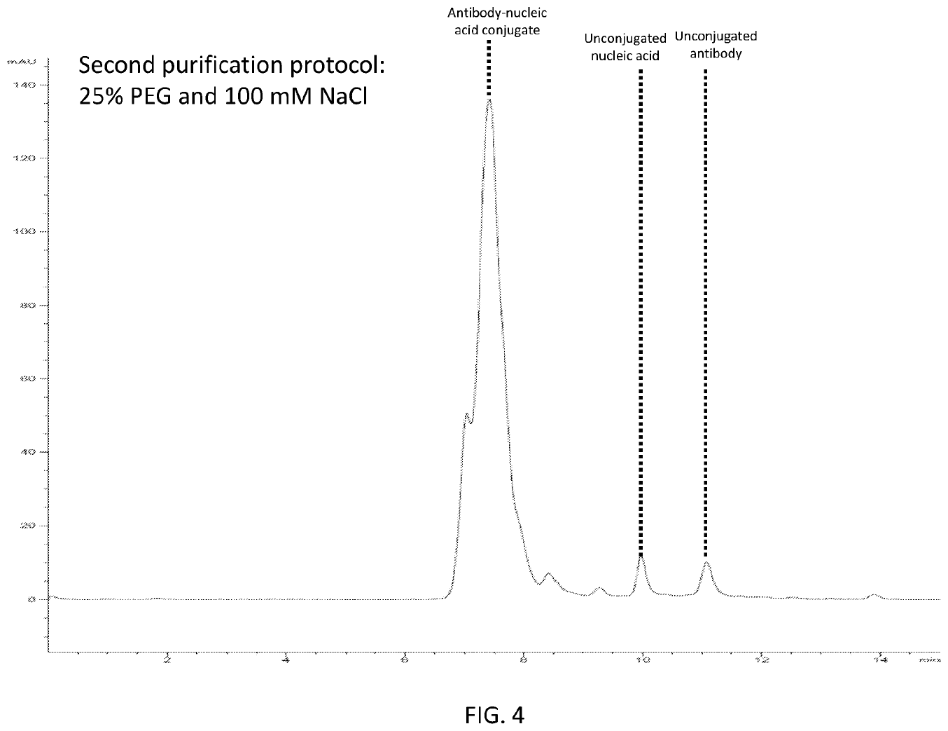

A conjugation reaction that was purified using the second purification protocol described above was analyzed using SEC HPLC. The results of this analysis are shown in FIG. 4. The horizontal lines in FIG. 4, from left to right, denote the expected elution times of the antibody-nucleic acid conjugate, the unconjugated nucleic acid and the unconjugated antibody. As shown in FIG. 4, the second purification protocol yielded a purified antibody-nucleic acid conjugate that was over 90% pure from the contaminating unconjugated nucleic acid.

Accordingly, the results described in this example demonstrate that the purification methods of the present disclosure, and more specifically the use of PEG and NaCl in the concentrations described herein, allow for the purification of antibody-nucleic acid conjugates to high levels of purity, including levels exceeding 90%.

### Example 2—Purification of Antibody-Nucleic Acid Conjugates Using the Methods of the Present Disclosure

The following is a non-limiting example that demonstrates that the purification methods of the present disclosure can be used to purify antibody-nucleic acid conjugates.

The antibody-nucleic acid conjugates were prepared as described in Example 1. These antibody-nucleic acid conjugates were then purified using one of two separate protocols.

In the first protocol, the conjugation reaction comprising the antibody-nucleic acid conjugates and the unconjugated nucleic acids was contacted with a purification solution comprising PEG-6000 and NaCL to produce a mixture comprising antibody-nucleic acid conjugates, unconjugated nucleic acids, PEG and NaCl such that the concentration of PEG in the mixture was about 25% (w/v) and the concentration of NaCl was about 100 mM. The mixture was then centrifuged to produce a supernatant and a pellet, the pellet was isolated from the supernatant, and the pellet was resuspended using a resuspension solution comprising PBS. These steps were then repeated twice to produce a purified solution of antibody-nucleic acid conjugates.

In the second protocol, the conjugation reaction comprising the antibody-nucleic acid conjugates and the unconjugated nucleic acids was contacted with a purification solution comprising PEG-6000 and NaCL to produce a mixture comprising antibody-nucleic acid conjugates, unconjugated nucleic acids, PEG and NaCl such that the concentration of PEG in the mixture was about 25% (w/v) and the concentration of NaCl was about 200 mM. The mixture was then centrifuged to produce a supernatant and a pellet, the pellet was isolated from the supernatant, and the pellet was resuspended using a resuspension solution comprising PBS. These steps were then repeated twice to produce a purified solution of antibody-nucleic acid conjugates.

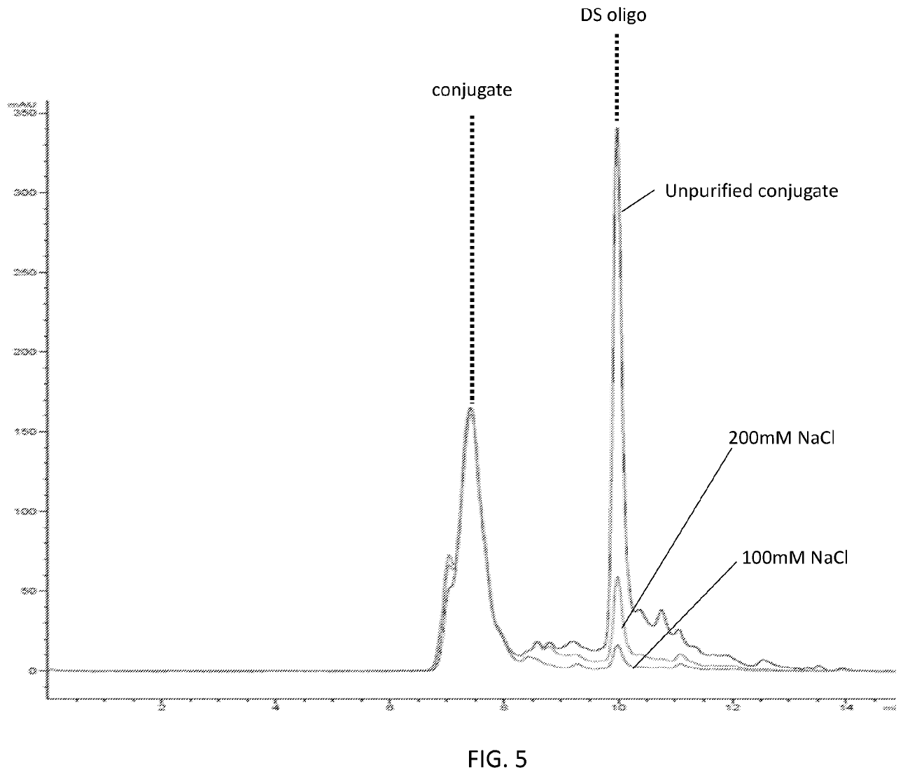

The solutions produced by each of the two purification protocols described above were then analyzed using SEC HLPC along with an aliquot of unpurified conjugation reaction to determine the extent of antibody-nucleic acid conjugation purification. The results of this analysis are shown in FIG. 5. The horizontal lines in FIG. 5, from left to right, denote the expected elution times of the antibody-nucleic acid conjugate, the unconjugated nucleic acid and the unconjugated antibody. As shown in FIG. 5, purification of the antibody-nucleic acid conjugate was achieved using both of the protocols.

Accordingly, the results described in this example demonstrate that the purification methods of the present disclosure, and more specifically the use of PEG and NaCl in the concentrations described herein, allow for the purification of antibody-nucleic acid conjugates to high levels of purity. Moreover, the results demonstrate that the concentration of NaCl can be varied within the ranges specified herein to optimize overall purity of the antibody-nucleic acid conjugate.

### Example 3—Antibody-Nucleic Acid Conjugates Purified Using the Methods of the Present Disclosure Demonstrate Unaltered Activity

The following is a non-limiting example that demonstrates the purification methods of the present disclosure do not alter the activity of the purified antibody-nucleic acid conjugates as compared to antibody-nucleic acid conjugates purified using existing SEC HPLC methods.

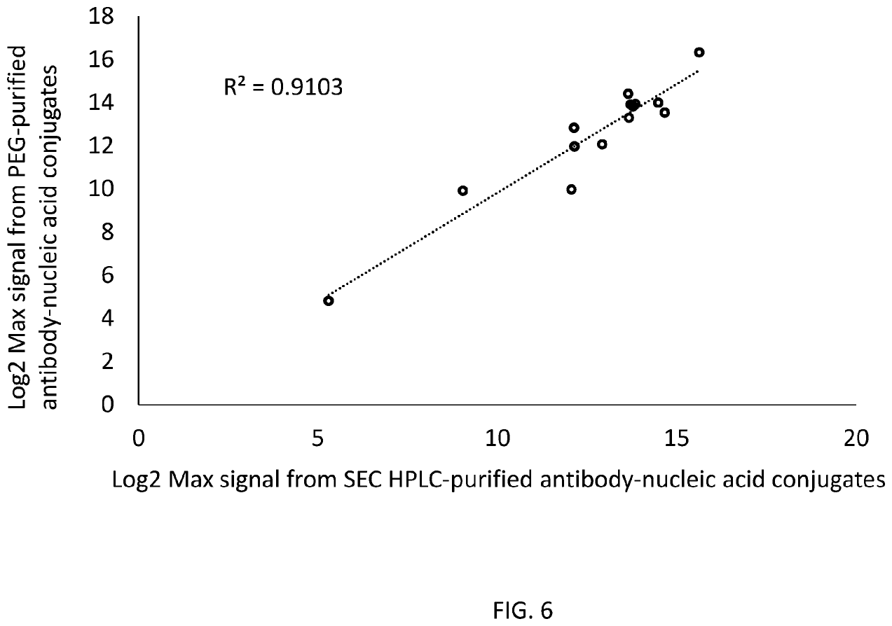

Fourteen different antibody-nucleic acid conjugates were purified using either standard SEC HPLC methods, or the PEG precipitation method described herein. The purified antibody-nucleic acid conjugates were then using in GeoMx assays performed on cell pellet arrays (see Merritt et al. Multiplex digital spatial profiling of proteins and RNA in fixed tissue. Nat Biotechnol. 2020 May;38(5):586-599 for information regarding GeoMx assays). The functional signals obtained in the assays using the SEC HPLC-purified antibody-nucleic acid conjugates and the PEG-purified antibody nucleic acid conjugates were then compared. This comparison is shown in FIG. 6. As shown in FIG. 6, the signals between the two purification methods were strongly correlated, indicated that the methods of the present disclosure do not impair the activity of the purified antibody nucleic acid conjugates.

### Example 4—Antibody-Nucleic Acid Conjugates Purified in Multi-Well Plates Using the Methods of the Present Disclosure

The following is a non-limiting example that demonstrates that the purification methods of the present disclosure can be performed in a multi-well (e.g. 96 well) plate, allowing for the rapid and simultaneous purification of several different species of antibody-nucleic acid conjugates.

Twelve different species of antibody-nucleic acid conjugates were purified using the PEG precipitation method described herein. Each species of antibody-nucleic acid conjugate comprised a different nucleic acid species and the antibody across all species of the generic polyclonal rabbit IgG. Each species was purified in an individual well of a 96-well plate. Table A shows the % yield of each of the different antibody-nucleic acid conjugates.

TABLE A

| Antibody-nucleic acid | |
|---|---|
| conjugate # | % Yield |
| 1 | 73 |
| 2 | 66 |
| 3 | 64 |
| 4 | 62 |
| 5 | 62 |
| 6 | 62 |
| 7 | 62 |
| 8 | 70 |
| 9 | 65 |
| 10 | 64 |
| 11 | 70 |
| 12 | 65 |
| Average | 65 |

### Example 5—Distinct Antibody-Nucleic Acid Conjugates Purified Using the Methods of the Present Disclosure

The following is a non-limiting example that demonstrates that the purification methods of the present disclosure is robust and can be used to purify various different antibody-nucleic acid conjugates, including those that differ in the identity of the antibody and oligonucleotide used.

Eight different species antibody-nucleic acid conjugates were purified using the PEG precipitation method described herein. Each species of antibody-nucleic acid conjugate comprised a different nucleic acid species and a different antibody species, as put forth in Table B. Each species was purified in a sample tube. Table B shows the % yield of each of the different antibody-nucleic acid conjugates.

TABLE B

| Antibody-nucleic acid conjugate | % Yield |
|---|---|
| Rat IgG2b - oligo 1 | 76 |
| Mouse IgG2b - oligo 2 | 61 |
| Rabbit IgG clone A - oligo 3 | 65 |
| Rabbit IgG clone B - oligo 4 | 62 |
| Rabbit IgG clone C - oligo 5 | 73 |
| Rabbit IgG clone D - oligo 6 | 74 |
| Rabbit IgG clone E - oligo 7 | 74 |
| Mouse IgG1 - oligo 8 | 72 |
| Average | 70 |

### Example 6—Pooled Purification Antibody-Nucleic Acid Conjugates Using the Methods of the Present Disclosure Demonstrate Unaltered Activity

The following is a non-limiting example that demonstrates the purification methods of the present disclosure can be performed “separate” format or in a “pooled” format and that the choice of format does not alter the activity of the purified antibody-nucleic acid conjugates. In this way, a plurality of different species of antibody-nucleic acid conjugates can be pooled and purified simultaneously using the methods of the present disclosure.

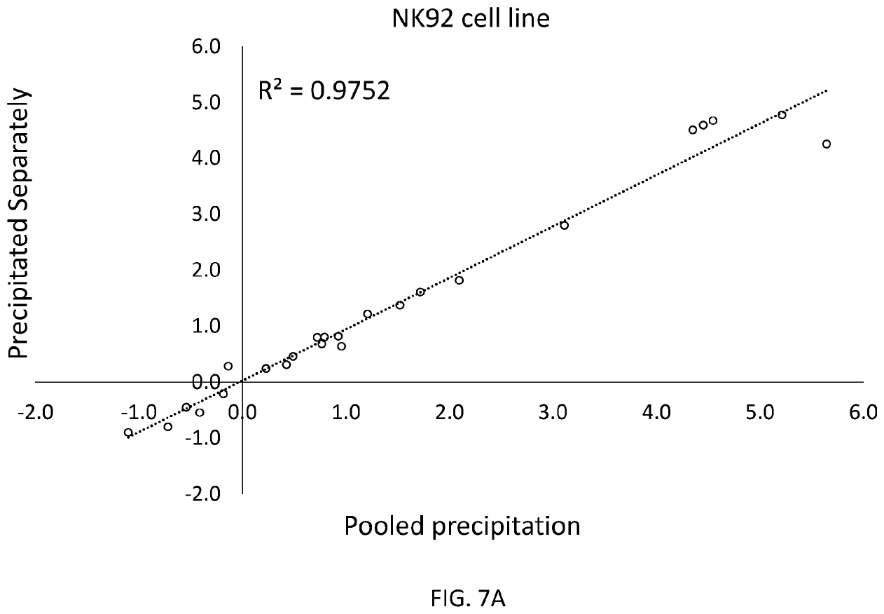

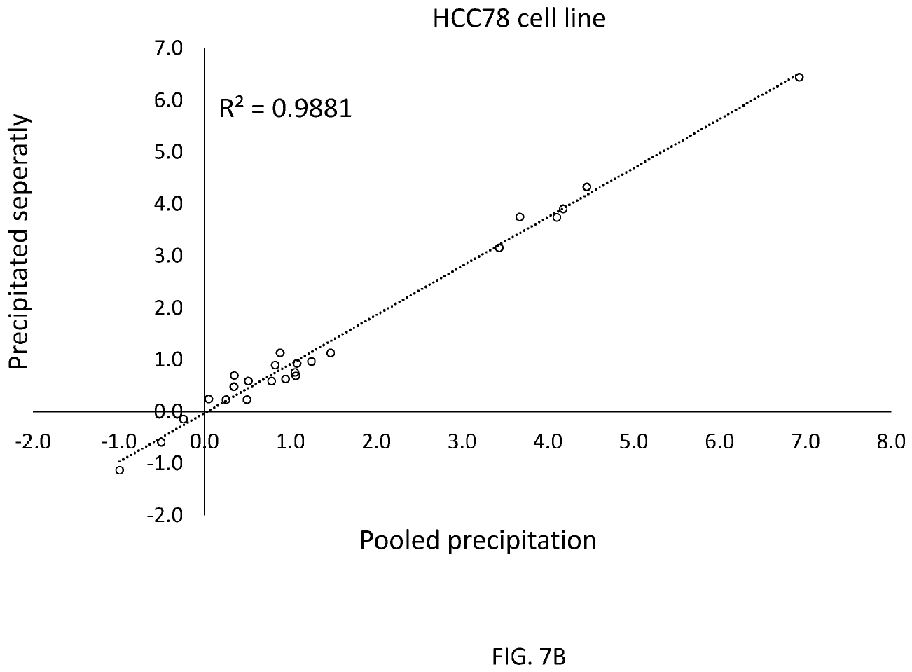

Over 20 different species of antibody-nucleic acid conjugates were purified separately (i.e. each species was purified in its own sample tube) or purified in a pooled format (i.e. the over 20 species were combined in a single sample tube prior to purification) using the PEG precipitation methods described herein. The antibody-nucleic acid conjugates were then used to assess two different cell lines, NK92 cells and HCC78 cells. The functional signals recorded using the separately purified or the pooled purified conjugates were then compared. This comparison is shown in FIGS. 7A and 7B. As shown in FIGS. 7A and 7B, the signals between the two purification formats were strongly correlated, indicating that the methods of the present disclosure can be used to separately purify different species of antibody-nucleic acid conjugates or purify a pool of a plurality of different species of antibody-nucleic acid conjugates.

What is claimed is:

1. A method for purifying antibody-nucleic acid conjugates from a solution including the antibody-nucleic acid conjugates and unconjugated nucleic acid, the method comprising:
a) contacting the solution and a purification solution including polyethylene glycol (PEG) and sodium chloride (NaCl) to produce a mixture including antibody-nucleic acid conjugates, unconjugated nucleic acids, PEG and NaCl such that the concentration of PEG in the mixture is between about 5% to about 30% (w/v) and the concentration of NaCl in the mixture is between about 100 mM to about 250 mM;
b) centrifuging the mixture to produce a supernatant and a pellet;
c) isolating the pellet from the supernatant;
d) resuspending the pellet with a resuspension solution to produce a purified solution;
e) repeating steps (a)-(d) at least once using the purified solution of step (d) to produce a purified solution that is at least 90% pure for the antibody-nucleic acid conjugates, as measured by size exclusion (SEC) high performance liquid chromatography (HPLC).

2. The method of claim 1, wherein the concentration of PEG in the mixture is between about 10% to about 25% (w/v).

3. The method of claim 1 or claim 2, wherein the concentration of NaCl in the mixture is between about 150 mM to about 200 mM.

4. The method of any one of the preceding claims, wherein the purified solution is at least 95% pure for the antibody-nucleic acid conjugates, as measured by size exclusion (SEC) high performance liquid chromatography (HPLC).

5. The method of any one of the preceding claims, wherein step (b) comprises centrifuging the mixture at a speed of at least about 1,000 RCF (xg), preferably wherein the centrifuging in step (b) is performed for at least about 2 minutes.

6. The method of any one of the preceding claims, wherein:
i) step (a) further comprises incubating the mixture for about 20 to about 40 minutes at about 20° C.; and/or
ii) step (d) further comprises incubating the resuspended pellet for about 20 to about 40 minutes at about 37° C.

7. The method of any one of the preceding claims, wherein the PEG is:
i) PEG-6000;
ii) PEG-8000;
iii) PEG-10000; or
iv) PEG-35,000.

8. The method of any one of the preceding claims, wherein contacting the solution and a purification solution in step (a) comprises contacting the solution and the purification solution at a ratio of 1:1 (v/v).

9. The method of any one of the preceding claims, wherein the concentration of PEG in the purification solution is between about 35% to about 45% (w/v), preferably wherein the concentration of PEG in the purification solution is about 40% (w/v).

10. The method of any one of the preceding claims, wherein the concentration of NaCl in the purification solution is between about 150 mM to 250 mM, preferably wherein the concentration of NaCl in the purification solution is about 200 mM.

11. The method of any one of the preceding claims, wherein the antibodies comprise a Fab, a Fab′, a F(ab′)2, a Fv fragment, a single-chain (sc)Fv (scFv) antibody fragment, a linear antibody, a single domain antibody (sdAb), a camelid VHH domain, a multi-specific antibody or any combination thereof.

12. The method of any one of the preceding claims, wherein the method is performed in one or more wells of a multi-well plate, thereby simultaneously purifying a plurality of antibody-nucleic acid conjugates.

13. The method of any one of the preceding claims, wherein the method is performed in two or more wells of a multi-well plate, thereby simultaneously purifying a plurality of antibody-nucleic acid conjugates in distinct wells.

14. A method of producing antibody-nucleic acid conjugates, the method comprising:
a) contacting a first solution comprising nucleic acids including at least one reactive group and a second solution including antibodies under conditions such that the at least one reactive group of the nucleic acids reacts with the antibodies, thereby forming antibody-nucleic acid conjugates;
b) purifying the antibody nucleic acid conjugates using the method of any one of claims 1-14.

15. The method of claim 14, wherein the at least one reactive group comprises a maleimide moiety, a iodoacetamide moiety, a benzylic halide moiety, a bromomethylketone moiety, an isothiocyanate moiety, a succinimidyl esters moiety, a carboxylic ester moiety, a sulfosuccinimidyl ester moiety, a 4-sulfotetrafluorophenyl (STP) ester moiety, a 2,4,5,6-Tetrafluorophenyl (TFP) ester moiety, a sulfodicholorphenol (SDP) ester moiety, a carbonyl azide moiety, a N-Hydroxysuccinimide (NHS) moiety, an azide moiety, an alkyne moiety, a tetrazine moiety, a aza-dibenzocyclooctyne moiety, a sulfonyl chloride moiety or any combination thereof

16. A kit comprising:
i) a first solution including nucleic acids including at least one reactive group; and
ii) a purification solution including polyethylene glycol (PEG) and sodium chloride (NaCl).

17. The kit of claim 16, wherein the at least one reactive group comprises a maleimide moiety, a iodoacetamide moiety, a benzylic halide moiety, a bromomethylketone moiety, an isothiocyanate moiety, a succinimidyl esters moiety, a carboxylic ester moiety, a sulfosuccinimidyl ester moiety, a 4-sulfotetrafluorophenyl (STP) ester moiety, a 2,4,5,6-Tetrafluorophenyl (TFP) ester moiety, a sulfodicholorphenol (SDP) ester moiety, a carbonyl azide moiety, a N-Hydroxysuccinimide (NHS) moiety, an azide moiety, an alkyne moiety, a tetrazine moiety, a aza-dibenzocyclooctyne moiety, a sulfonyl chloride moiety or any combination thereof.

18. The kit of claim 16 or claim 17, further comprising a second solution including antibodies.

19. The kit of claim 18, wherein the antibodies comprise a Fab, a Fab′, a F(ab′)2, a Fv fragment, a single-chain (sc)Fv (scFv) antibody fragment, a linear antibody, a single domain antibody (sdAb), a camelid VHH domain, a multi-specific antibody or any combination thereof.

20. The kit of any one of claims 16-19, further comprising a third solution including nucleic acids including at least one reactive group,
wherein the nucleic acids in the first solution include a nucleic acid sequence that is different from the nucleic acid sequence of the nucleic acids in the third solution.

21. The kit of any one of claims 16-20, wherein the PEG is:
i) PEG-6000;
ii) PEG-8000;
iii) PEG-10000; or
iv) PEG-35,000.

22. The kit of any one of claims 16-21, wherein the concentration of PEG in the purification solution is between about 35% to about 50% (w/v), preferably wherein the concentration of PEG in the purification solution is about 40% (w/v).

23. The kit of any one of claims 16-22, wherein the concentration of NaCl in the purification solution is between about 150 mM to 250 mM, preferably wherein the concentration of NaCl in the purification solution is about 200 mM.

24. The kit of any one of claims 16-23, wherein the kit further comprises at least one of:
i) at least one aliquot of TCEP
ii) at least one spin filter, and
iii) at least one multi-well plate.
